(19) United States
(12) Patent Application Publication
(10) Pub. No.: US 2011/0207163 A1
(43) Pub. Date: Aug. 25, 2011

(54) METHOD FOR AND MATERIAL OF A SHAPE STANDARD

(75) Inventor: Dietmar Lerche, Berlin (DE)

(73) Assignee: Dr. Lerche KG, Berlin (DE)

(21) Appl. No.: 12/996,517

(22) PCT Filed: Jun. 4, 2009
(86) PCT No.: PCT/EP09/56872

§ 371 Date: Apr. 29, 2011

(51) Int. Cl. C12Q 1/02; C12Q 1/00; G01Q 60/00

(52) U.S. Cl. 435/29; 435/4; 850/21

(57) ABSTRACT

A method is disclosed for the preparation of isomorphic and nonspherical shape standards as isometric as possible and their mixtures for validation of the measurement methods and analyzers for quantification of particle shapes and their characteristics especially in the microscale and nanoscale size range. Furthermore the shape standards also provide quality control and performance qualification can be used at the operator of the corresponding shape measurement devices.

Macroscale, microscale, and nanoscale particles play an important part in development, production and processing in process engineering, biotechnology, the food industry or pharmacy. In addition to the particle size, the particle shape is being increasingly assessed and quantified by means of innovative measurement techniques. Therefore, for the manufacturer of the corresponding measurement hardware first the object is to validate the developed measurement technologies and especially the mathematical algorithms, second, from the standpoint of the users the question of comparability, sensitivity and reproducibility of different measurement techniques is of great importance, and third, not only in a controlled market quality management systems demand performance qualification of the measurement hardware used. Novel shape-defined reference particles are required for this purpose.

Liquid-liquid, liquid-solid or solid-liquid dispersions (for example, emulsions, suspensions or suspoemulsions) macroscale, microscale, and nanoscale particles play an important part in development, production and processing in process engineering, biotechnology, the food industry or pharmacy and are used in all spheres of life. Here stronger and stronger functionalization of particles occurs which also takes place in part by special shaping. Furthermore flow and abrasion characteristics depend largely on the shape. Since the determination of the grain size has been in the foreground in the R&D sphere and in industry in the last half century, with the increase of the quality requirements for innovative solutions and products, demands for quantification of the particle shape and aggregate state have increased greatly. Last but not least, this is documented by elaborating a new international standard ISO/FDIS 9276-6 which defines the necessary characteristics. By way of example, here for instance only Feret diameter, length-width ratio, circle- and ellipse-equivalent diameter, and the great circle are named as macrodescriptors and for example sphericity, angularity, concavity, convexity are named as mesodescriptors.

Therefore, in recent years a plurality of analytic instruments have been developed and marketed for shape analysis of particles mainly in the microscale size range. Very different measurement methods are used, such as for example application of particles to a measurement surface and digital photography with and without aids for enlarging the objects (for example ITS Technologies AG, Vilters), dispersion of particles in a gaseous carrier medium and recording the particles sinking past the recording optics in free fall in a measurement shaft (for example Nanophox, Sympatec GmbH, Clausthal; CamSizer, Retsch Technology GmbH, Hahn), dispersion of particles in a liquid and recording the particles which are flowing through a hydrodynamically focused or unfocused measurement volume (for example, FlowCam, Fluid Imaging Technologies Inc. Yarmouth, Me.). Methods are also known which for purposes of chord length measurement detect the interruption of a laser beam by particles moving past (Laser Obscuration Time (LOT) Technology, Ankersmid, Netherlands). Finally, developments which measure the angle-dependent scattering intensity of the incident laser light for shape analysis are also on the market.

The acquisition of micro descriptors in general presupposes the improvement of the measurement technique and is of rather subordinate importance for the object of this invention.

Without detailing technical problems of direct detection of the shape-dependent primary signal, all methods must use mathematical algorithms which convert the experimentally determined signals into a three-dimensional shape of the measured objects and indicate the particle quantity statistics with respect to fractions with different shape and size. The complexity of this object becomes apparent if it is considered that in many technical products the particles differ both in their volume and also in their three-dimensional shape and the ascertained primary signal is generally only one- or two-dimensional.

First of all, for the manufacturers of the corresponding measurement hardware the object is to validate the developed measurement technology and especially the mathematical algorithms, second, from the standpoint of the users the question of comparability, sensitivity and reproducibility of various measurement techniques is of great importance, and third, quality management systems demand performance qualification of the measurement hardware used not only in a regulated market.

These objects require suitable shape standards. To date there have not been either certified or uncertified isomorphic reference particles (except for spherical ones which cannot be used for this object) with which the computation algorithms can be tested, hardware qualification can be enabled and different devices of the same type or different measurement methods can be compared.

One reason for this is that the economical production of strictly isomorphic nonspherical small particles is technically extremely difficult and even with crystallization a broad distribution both of shape parameters and also numerical characteristics occurs and thus the demands for reference material cannot be satisfied. Classification methods with respect to shape are unknown.

Therefore the object of the invention is a method for and the preparation of isomorphic and nonspherical shape standards as isometric as possible and their mixtures for validation of the measurement methods and analyzers for quantification of particle shapes and their characteristics especially in the microscale and nanoscale size range. Furthermore the shape standards as claimed in the invention, also quality control and performance qualification will be used at the operator of the corresponding shape measurement devices.

This object was achieved according to the features of the claims.

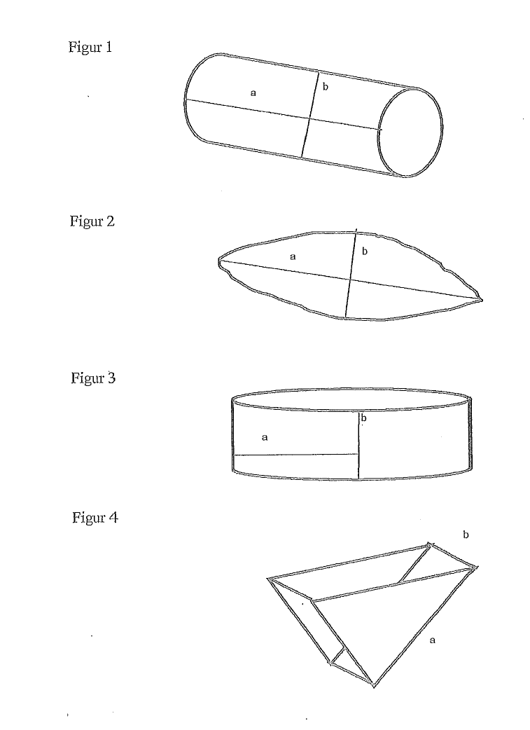

Interestingly, nature offers just these isomorphic objects. Thus for example seeds of many plants are characterized by a rather large diversity of shapes (cylinder shape, ellipsoids, kidney shape) (Seed Atlas of the Most Important Forage Plants and Their Weeds, Deut. Bauerverlag, Berlin, 1955; Wonderful Plant World. Seeds and Fruits. Stuttgart Parkland 1995, ISBN: 38880597960). For example for diatoms, spores or plant pollen there is very great diversity of shapes with very low variability of the shape characteristics. The inventive approach therefore consists in identifying suitable objects and using just these objects as a starting point for technically usable shape standards. Advantageously there are two aspects here. On the one hand, the biological objects are very isomorphic (same shape), and secondly very isometric (quantitatively identical characteristics). For example, pollens are named here which are characterized by great similarity with respect to shape and size (FIGS. 1 to 5) for the respective species in spite of a great diversity of shape for different species. Due to the diversity and constancy of shape of very isomorphic objects, after corresponding processing, particles with different morphological characteristics can ideally be made available in order to thus test the algorithms and sensitivity of the measurement methods under consideration with respect to different shape indices and to compare different technological measurement methods. It is also advantageous that for example seeds are established in the millimeter range, pollen and cells in the micron range and spores in the nanometer range and thus shape standards with different equivalent quantities for different measurement methods according to their size resolution can be made available. Surprisingly, biological objects have also been found which can be changed gradually in their shape by simple treatment and afterwards this shape can be “frozen in” by chemical treatment, for example with glutaric aldehyde. One example is the mammal erythrocyte. The typical dented disc-like base shape is shown schematically in FIG. 6. The volume of these cells in the isomorphic shape varies very greatly in a noteworthy manner, for example 87 fl (man), 50 fl (cattle) and 31 fl (sheep). The volume is characterized by a high constancy (standard deviation in healthy human erythrocyte roughly 10%). By increasing the osmotic pressure of the suspension medium the normal discoid shape (sphericity index 0.78) can be further dented or by diminution the shape can be continuously swollen as far as a spherical configuration (sphericity index 1.0) (Meier et al. Studia biophysica, 1983, 93, 101-109). Thus, different shapes can be gradually produced in a simple manner, fixed with glutaric aldehyde and the sensitivity of the mathematical algorithms used in the devices can be checked and different hardware technologies and evaluation approaches can be compared.

It is also possible for example by adsorbing or nonadsorbing polymers to produce aggregates and agglomerates in a controlled manner from the individual particles and thus to further greatly increase the diversity of shape.

It has also been interestingly shown that the surface of the objects (for example, pollen from lillies) can have a very pronounced structure and thus also the sensitivity of the measurement methods on surface structures and the evaluation of mesodescriptors and microdescriptors can be tested.

According to the different measurement techniques, it becomes necessary for the shape standard particles to be used dispersed dry or wet. In the case of producing suspensions, the variation of the particle volume concentration or mass concentration can be additionally of interest. It is especially advantageous that different isomorphic standard particles can be mixed in any ratios regardless of their dispersion shape and thus bimodal, trimodal, and polymodal isomorphic and/or isometric test samples are available.

It has furthermore been shown that by modifying the surface for example by binding a dye or coating with a material with suitable index of refraction the use of low-contrast shape standards for example for flow-optical methods can be improved.

To obtain the starting material, methods can advantageously be used which for example were developed for blood cells in transfusiology, for obtaining pollen as food additives or allergenic test material, for the cultivation and harvesting of diatoms, the harvesting of plant seeds etc. For the inventive approach high demands must be imposed on species purity. Generally the starting biological material which has been obtained must be cleaned and optionally classified. It is advantageous that naturally these objects are often present dry dispersed. It has proven beneficial that for example seeds, pollen or spores can be dried to a species-dependent residual moisture content and when this is maintained during storage, long usability is guaranteed. Additional treatment with chemicals for inhibiting metabolism or insecticides, fungicides, etc. benefits the quality of the shape standards stored dry and storage times of years are possible. It is also advantageously possible to store the initial objects, intermediate products or the final reference shape standards cooled (for example 4° C.) or quick-frozen (for example 18° C.) and thus to lengthen the storage times to years.

Liquid dispersion proceeds from dry objects or fixed biological objects. Here the dispersing liquids should be chosen such that the dry objects do not dissolve, shrink or swell in the respective liquid. Often the use of nonaqueous, low-viscosity dispersing media, for example nonaqueous silicone oil or alcohol, has proven advantageous.

For the gradual change of the shape, media which allow the object to swell or shrink can be used in a dedicated matter. Thus mammal cells can be changed in a controlled manner in their shape by Ringer solutions with set nonphysiological osmotic pressure. The resulting shape can be stored for years with constant shape, stabilized and dispersed wet advantageously for example by fixing with glutaric aldehyde.

In the collection of objects and in the dispersion it must be generally watched that particles are not damaged or, if not expressly desired, cementing, aggregation or coagulation of individual particles does not occur. The latter or naturally occurring aggregates or clumps can be advantageously suppressed by corresponding processing steps (for example washing) and/or the use of surfactants, dispersants and stabilizers. In particular, in wet dispersion the addition of antibiotics which prevent for example bacterial decomposition of the reference particles benefits the storageability of the sample.

In some cases it has however proven advantageous to induce aggregation of particles in certain media or after surface modification (for example coupling of receptors) aggregate formation in a controlled manner and thus to obtain larger secondary particles based on isomorphic primary particles. Surface modification without aggregate formation can also be advantageous when the shape standard for special measurement methods can only be used in this way. The coloring or application of a reflection layer was successfully practiced as one example of treatment.

It has been shown that recovery of larger amounts of the parent material is not always given. In these cases, by blending of several parent samples a representative lot amount can be obtained and processed accordingly. Ascertaining the shape characteristics by means of a reference method (for example scanning microscopy) can be done by taking representative samples from the entirety. For example, some determined characteristics for 5 different standards are given in FIGS. 1 to 5.

Then the entire lot is advantageously prepared with a corresponding sample dividing technology (for example, riffler) and corresponding sample vessels filled with it such that the sample can be supplied later to the measurement method to be validated without further preparation (ready to use).

The material which can be produced with the approach as claimed in the invention for shape standards is also especially well suited to mixing several different isomorphic reference particles (samples) in any amounts and thus to preparing polyisomorphic test samples with known composition. Here, depending on the special measurement technique the particle amount(s) can be set both as mass concentration, volume concentration or number concentration.

The samples which are characterized with respect to their shape and the distribution of shape characteristics with a reference method which should be linked to a national standard were subjected to shape analysis with the PowerShape (IST AG) system according to the instructions of the hardware manufacturer. For this purpose, by way of example dry discoid shape standards were distributed manually on the recording surface and the shape of the particles was recorded digitally by means of a scanner (4000 dpi). Then, by means of current software, from 3125 particles the shape descriptors convexity (0.9376), ellipticity (1.6220) or crystallinity (1.1338) as well as the grain size (21.13 μm) were determined. The standard deviations of the shape values were between 22.4% and 3.9%. It follows from experimental values that the evaluation algorithms work in a fundamentally stable manner and enable meaningful reclassification, but the alignment of the reference particles on the recording surface however influences the result and thus higher standard deviations result. A further test experiment was run with a wet dispersion with the Image-Pro Plus system. In this case the particles were dispersed in an alcohol-water mixture and the concentration of the reference particles was set according to production data. In this case for example, among others, the length, width, perimeter, the minimum Feret diameter, the maximum Feret diameter, the roundness and compactness of 50 particles were determined. The maximum standard deviation of all shape descriptors in this case (dynamic image evaluation) was only 5.9%. Length and width agreed very well with the reference values. It was furthermore shown that the particles swell in the aforementioned dispersion medium. Thus the roundness immediately after dispersion was 0.52, and after 11 days, 0.74. The swelling process can be controlled by the amount of water.

Explanation of FIGS. 1 to 5

FIG. 1:

| | | Length [μm] | Diameter [μm] |
|---|---|---|---|
| Shape | | a | b |
| Cylindrical Pollen Shape | MW | 29.82 | 14.80 |
| | Rod Width | 1.34 | 1.17 |
| | n | 42.00 | 41.00 |

FIG. 2:

| | | Perimeter [μm] | Perimeter [μm] |
|---|---|---|---|
| Shape | | a | b |
| Spindle-Shaped Pollen | MW | 69.94 | 35.82 |
| Shape | Rod Width | 4.30 | 2.93 |
| | n | 12 | 12 |

FIG. 3:

| | | Radius [μm] | Height [μm] |
|---|---|---|---|
| Shape | | a | b |
| Discoid Pollen Shape | MW | 22.79 | 19.13 |
| | Rod Width | 1.61 | 1.39 |
| | n | 49.00 | 19.00 |

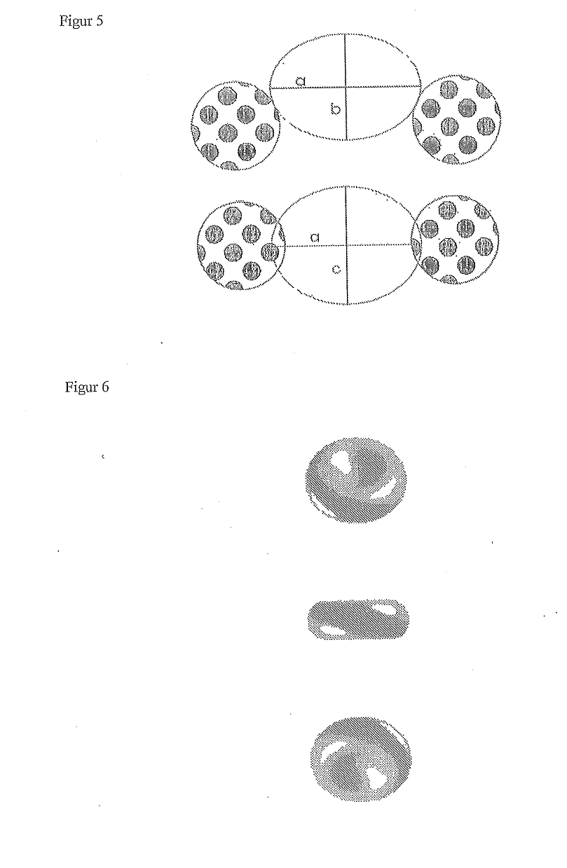

FIG. 4:

| | | Edge Length [μm] | Height [μm] |
|---|---|---|---|
| Shape | | a | b |
| Prismatic Pollen Shape | MW | 24.71 | 14.31 |
| | Rod Width | 1.58 | 2.03 |
| | n | 33.00 | 7.00 |

FIG. 5:

| Shape: | Perimeter | Perimeter | Perimeter | |
|---|---|---|---|---|
| Combined | [μm] | [μm] | [μm] | V/A |
| Pollen Shapes | a | b | c | [μm] |
| Pinus nigra | 41 ± 4.1 | 29 ± 3.0 | 32 ± 3.1 | 5.58 |
| n | 123 | 86 | 38 | |
| Pinus sylve-stris | 37 ± 3.5 | 26 ± 2.4 | 29 ± 3.5 | 5.01 |
| n | 129 | 90 | 41 | |

FIG. 6:

The typical dented disc-like base shape (eryshape) is shown schematically in FIG. 6.

1. Method for validation and quantification of particle shapes and their characteristics in a microscale or nanoscale size range, comprising:
quantifying a parent material which includes different biological objects with nonspherical shapes using an independent measurement method with respect to characteristics which are relevant to a shape description as a shape standard;
analyzing the shape standard or a mixture of different shape standards with the measurement method; and the
comparatively evaluating a result of the quantifying and analyzing.

2. Method as claimed in claim 1, wherein the biological objects can be seeds, fruits, pollen, spores, algae, cells with different nonspherical shape, or different particle volumes and particle masses.

3. Method as claimed in claim 1, herein the biological objects differ with respect to mesodescriptors and microdescriptors.

4. Method as claimed in claim 1, wherein the objects are stabilized in their original shape and/or are gradually modified by treatment; and afterwards stabilized.

5. Method as claimed in claim 1, comprising:
controlled alteration of a surface of the parent material.

6. Method as claimed in claim 1, wherein both dry-dispersed and wet-dispersed shape standards are used.

7. Method as claimed in claim 1, wherein mixtures of different shape standards are used.

8. Method as claimed in claim 1, comprising:
producing the parent material from natural biological sources and/or controlled cultivation and by collection, harvesting or isolation.

9. Method as claimed in claim 8, comprising:
purifying and classifying the parent material according to its shape, its volume or its mass.

10. Method as claimed in claim 8, wherein surface treatment takes place or primary objects are aggregated or agglomerated.

11. Method as claimed in one of claim 8, wherein the parent material is dried and preserved over a time interval by deactivation of metabolism.

12. Method as claimed in one of claim 8, wherein the parent material is dispersed wet in aqueous or nonaqueous fluids and is stabilized or preserved over a time interval by additives and/or by rigidification.

13. Method as claimed in claim 8, comprising:
varying a volume or mass in a controlled manner while maintaining isomorphism by corresponding cultivation or growing conditions, and the volume or the mass and morphology is gradually changed.

14. Method as claimed claim 8, wherein production of the parent material takes place from a large standard parent amount or by blending of different batches/lots, and the shape-relevant characteristics of the respective shape standard after corresponding production/processing is statistically ascertained in a reliable manner by a reference method.

15. Method as claimed in claim 14, wherein a measured master batch of the parent material is prepared and decanted such that an amount of a sample and form of availability does not require sample preparation a user.

16. method according to claim 4, wherein the treatment is swelling, shrinking or formation of aggregates or agglomerate, in a controlled manner.

17. Method according to claim 10, wherein the primary objects are aggregated or agglomerated by absorbing or non absorbing polymers.

18. Method according to claim 11, wherein the deactivation includes an addition of insecticides or fungicides.

19. Method according to claim 12, wherein the additives are dispersant aids or antibiotics.

20. Method according to claim 13, wherein the cultivation or growing conditions include light intensity or nutrient supply.
